(19) United States
(12) Patent Application Publication
(10) Pub. No.: US 2005/0232387 A1
(43) Pub. Date: Oct. 20, 2005

(54) Microfluidic apparatus and method for synthesis of molecular imaging probes

(76) Inventors: Henry C. Padgett, (US); Charles Russell Buchanan, (US); Thomas Lee Collier, (US); Joseph C. Matteo, (US); Charles W. Alvord, (US)

Correspondence Address: PITTS AND BRITTIAN P C, P O BOX 51295, KNOXVILLE, TN, 37950-1295, US

(21) Appl. No.: 10/827,893

(22) Filed: Apr. 20, 2004

(51) Int. Cl. A61M 36/14

(52) U.S. Cl. 376/194

(57) ABSTRACT

The invention provides a method and apparatus for preparation of radiochemicals, such as PET molecular imaging probes, wherein the reaction step or steps that couple the radioactive isotope to an organic or inorganic compound to form a positron-emitting molecular imaging probe are performed in a microfluidic environment. The method for synthesizing a radiochemical in a microfluidic environment comprises: i) providing a micro reactor comprising a first inlet port, a second inlet port, an outlet port, and at least one microchannel in fluid communication with the first and second inlet ports and the outlet port; ii) introducing a reactive precursor into the first inlet port of the micro reactor, the reactive precursor adapted for reaction with a radioactive isotope to form a radiochemical; iii) introducing a solution comprising a radioactive isotope into the second inlet port of the micro reactor; iv) contacting the reactive precursor with the isotope-containing solution in the microchannel of the micro reactor; v) reacting the reactive precursor with the isotope-containing solution as the reactive precursor and isotope-containing solution flow through the microchannel of the micro reactor, the reacting step resulting in formation of a radiochemical; and vi) collecting the radiochemical from the outlet port of the micro reactor.

### BACKGROUND OF THE INVENTION

1. Field of the Invention

The invention relates to the use of microfluidic devices and methods for chemical synthesis, particularly the use of microfluidic devices and methods for the synthesis of positron-emitter labeled PET molecular imaging probes.

2. Description of the Related Art

Positron Emission Tomography (PET) is a molecular imaging technology that is increasingly used for detection of disease. PET imaging systems create images based on the distribution of positron-emitting isotopes in the tissue of a patient. The isotopes are typically administered to a patient by injection of probe molecules that comprise a positron-emitting isotope, such as F-18, C-11, N-13, or O-15, covalently attached to a molecule that is readily metabolized or localized in the body (e.g., glucose) or that chemically binds to receptor sites within the body. In some cases, the isotope is administered to the patient as an ionic solution or by inhalation. One of the most widely used positron-emitter labeled PET molecular imaging probes is 2-deoxy-2-[¹⁸F]fluoro-D-glucose ([¹⁸F]FDG).

Since the inception of PET imaging in the late 1970's, PET radiochemical synthesis systems have used standard bench-top synthesis techniques, multi-milligram and multi-milliliter quantities of reagents, and multi-gram quantities of purification media, along with macro-scale reaction vessels and relatively large valve-and-tubing processing hardware.

The specific activity of the labeled molecular imaging probe is particularly sensitive to the relatively large scale of known synthesis processes. The specific activity of an isotope or molecular imaging probe is the amount of radioactivity relative to the mass, often given in Curie/mole (or Becquerel/mole). The mass consists of all isotopic forms of the radioactive label. The addition of a stable isotope along with the radioactive isotope will result in a dilution or lowering of the specific activity. Examples of lowered specific activity are the dilution of C-11 with stable C-12, or the addition of stable F-19 to F-18.

The maximum specific activity for fluorine-18 is 1,710 Ci/μmol, and for carbon-11 it is 9,240 Ci/μmol. [¹⁸F] fluoride ion produced by proton bombardment of a metal target filled with [¹⁸O] water in a cyclotron typically has a specific activity of about 50-100 Ci/μmol. This represents up to a 40 to 1 dilution with stable fluorine-19 that is present in the [¹⁸O] water, and released from the metal target body and polymeric valves and tubing in the target delivery system. In general, ¹⁸F-labeled molecular imaging probes prepared from [¹⁸F] fluoride ion have a specific activity of about 2-5 Ci/μmol after coupling the ion to a probe molecule, which means that the radiochemical synthesis process results in another 25 to 1 dilution with stable fluorine-19. Fluoride ion delivered from the cyclotron target will typically contain 0.2-0.4 μg (10-20 μmol) stable [¹⁹F] fluoride ion along with the radioactive [¹⁸F] fluoride ion. If the activity delivered is 1.0 Ci, the [¹⁸F] fluoride ion mass will be about 9.0 ng or 0.5 nmol. The same issues arise when using carbon-11 or other radioactive isotopes because the prior art radiochemical synthesis processes are the major source of unwanted carbon-12 or other stable isotopes.

U.S. Pat. No. 4,794,178, which is incorporated by reference herein in its entirety, discloses a process for producing ¹⁸F labeled organic compounds by nucleophilic substitution.

In the case of F-18, by using various trapping techniques either with an anion resin or with electroplating, the fluoride ion can be separated from the bulk target water.

There is a need in the art of radiochemical synthesis for devices and methods that produce radiochemicals, exhibiting faster synthesis times, and higher synthesis yields.

### SUMMARY OF THE INVENTION

The present invention provides a method and apparatus for preparation of radiochemicals, such as PET molecular imaging probes, wherein the reaction step or steps that couple the radioactive isotope to an organic or inorganic compound to form a positron-emitting molecular imaging probe are performed in a microfluidic environment (i.e., a micro reactor). The reaction(s) to form the radiolabeled molecular imaging probes can utilize gaseous or liquid reagents in a liquid/liquid phase, liquid/gas phase or gas/gas phase reaction. The use of microfluidics and micro reactor technology for the radiochemical synthesis of labeled molecular imaging probes is advantageous because it matches the scale of the synthesis equipment and techniques to that of the radioactive labeling reagents, thereby promoting faster synthesis times, and higher synthesis yields. These systems are small, simple, reliable, microfluidics-based radiochemical synthesis systems.

In one aspect, the invention provides a method for synthesizing a radiochemical in a microfluidic environment, the method comprising: i) providing a micro reactor comprising a first inlet port, a second inlet port, an outlet port, and at least one microchannel in fluid communication with the first and second inlet ports and the outlet port; ii) introducing a reactive precursor into the first inlet port of the micro reactor, the reactive precursor adapted for reaction with a radioactive isotope to form a radiochemical; iii) introducing a solution comprising a radioactive isotope into the second inlet port of the micro reactor; iv) contacting the reactive precursor with the isotope-containing solution in the microchannel of the micro reactor; v) reacting the reactive precursor with the isotope-containing solution as the reactive precursor and isotope-containing solution flow through the microchannel of the micro reactor, the reacting step resulting in formation of a radiochemical; and vi) collecting the radiochemical from the outlet port of the micro reactor.

Preferably, the radioactive isotope and reactive precursor are dissolved in a polar aprotic solvent and moved through the micro reactor using at least one syringe or other suitable pump. The reactive precursor and isotope-containing solution are preferably heated during the reacting step. In one embodiment, the micro reactor comprises a first microchannel segment in fluid communication with the first inlet of the micro reactor, a second microchannel segment in fluid communication with the second inlet of the micro reactor, and a third microchannel segment in fluid communication with the outlet of the micro reactor, wherein the first, second and third microchannel segments intersect. In preferred embodiments, the above method further comprises performing at least one additional method step in a microfluidic environment, such as deprotecting the radiochemical, purifying the radiochemical, and/or assaying radioactivity of the radiochemical.

In a particularly preferred embodiment of the method described above, a fluorine-18 fluoride labeled radiochemical is synthesized in a microfluidic environment using a method comprising the steps of: i) providing a micro reactor comprising a first inlet port, a second inlet port, an outlet port, and at least one microchannel in fluid communication with the first and second inlet ports and the outlet port; ii) introducing a liquid organic reactive precursor dissolved in a polar aprotic solvent into the first inlet port of the micro reactor, the organic reactive precursor adapted for reaction with fluorine-18 fluoride to form a radiochemical; iii) introducing a solution comprising fluorine-18 fluoride dissolved in a polar aprotic solvent into the second inlet port of the micro reactor; iv) contacting the organic reactive precursor with the isotope-containing solution in the microchannel of the micro reactor; v) reacting the organic reactive precursor with the fluorine-18 fluoride solution in a nucleophilic substitution reaction as the reactive precursor and fluorine-18 fluoride solution flow through the microchannel of the micro reactor, the reacting step resulting in formation of a fluorine-18 fluoride labeled radiochemical; and vi) collecting the fluorine-18 fluoride labeled radiochemical from the outlet port of the micro reactor.

Particularly preferred fluorine-18 fluoride ion labeled radiochemicals include 2-deoxy-2-[¹⁸F]fluoro-D-glucose([¹⁸F]FDG), 6-[¹⁸F]fluoro-L-3,4-dihydroxyphenylalanine([¹⁸F]FDOPA), 6-[¹⁸F]fluoro-L-meta-tyrosine([¹⁸F]FMT), [¹⁸F]fluorocholine, [¹⁸F]fluoroethylcholine, 9-[4-[¹⁸F]fluoro-3-(hydroxymethyl)butyl]guanine([¹⁸F]FHBG), 9-[(3-[¹⁸F]fluoro-1-hydroxy-2-propoxy)methyl]guanine([¹⁸F]FHPG), 3-(2′-[¹⁸F]fluoroethyl)spiperone([¹⁸F]FESP), 3′-deoxy-3′-[¹⁸F]fluorothymidine([¹⁸F]FLT), 4-[¹⁸F]fluoro-N-[2-[1-(2-methoxyphenyl)-1-piperazinyl]ethyl]-N-2-pyridinyl-benzamide([¹⁸F]p-MPPF), 2-(1-{6-[(2-[¹⁸F]fluoroethyl)(methyl)amino]-2-naphthyl}ethylidine)malononitrile([¹⁸F]FDDNP), 2-[¹⁸F]fluoro-α-methyltyrosine, [¹⁸F]fluoromisonidazole([¹⁸F]FMISO), 5-[¹⁸F]fluoro-2′-deoxyuridine([¹⁸F]FdUrd), [¹¹C]raclopride, [¹¹C]N-methylspiperone, [¹¹C]cocaine, [¹¹C]nomifensine, [¹¹C]deprenyl, [¹¹C]clozapine, [¹¹C]methionine, [¹¹C]choline, [¹¹C]thymidine, [¹¹C]flumazenil, [¹¹C]β-aminoisobutyric acid ([¹¹C]β-AIBA), and other small physiologically-active molecules that are labeled using fluoride ion and protected forms thereof.

In another aspect, the invention provides a system for synthesizing a radiochemical in a microfluidic environment, the system comprising a micro reactor comprising a first inlet port, a second inlet port, an outlet port, and at least one microchannel in fluid communication with the first and second inlet ports and the outlet port; a supply of a reactive precursor in fluid communication with the first inlet port of the micro reactor, the reactive precursor adapted for reaction with a radioactive isotope to form a radiochemical; and a supply of a solution comprising a radioactive isotope in fluid communication with the second inlet port of the micro reactor. Preferably, the system further includes at least one pump (e.g., a syringe pump or other suitable pump) operatively positioned to propel the reactive precursor and the isotope-containing solution through the micro reactor. In one embodiment, the system includes a first pump in fluid communication with the supply of reactive precursor and the first inlet of the micro reactor and a second pump in fluid communication with the supply of isotope-containing solution and the second inlet of the micro reactor. The system may further include a heat source operatively positioned to heat at least a portion of the micro reactor. The micro reactor may comprise, for example, a microchip comprising a substrate having at least one microchannel formed therein or a length of capillary tubing defining at least one microchannel.

### BRIEF DESCRIPTION OF THE DRAWINGS

FIG. 1 is a schematic representation of a PET molecular imaging probe synthesis process;

FIG. 2 is a schematic representation of an embodiment of a microfluidic radiochemical synthesis apparatus according to the present invention;

FIG. 3 is a schematic representation of another embodiment of a microfluidic radiochemical synthesis apparatus according to the present invention comprising two microchips connected in series;

FIG. 4 is a schematic representation of a syringe or other suitable pumping system suitable for use in the microfluidic system of the invention;

FIG. 5 is a schematic representation of a further embodiment of a microfluidic radiochemical synthesis apparatus according to the present invention with integrated microfluidic reagent reservoirs;

FIG. 6 is a schematic representation of a further embodiment of a microfluidic radiochemical synthesis apparatus according to the present invention in fluid communication with the target body;

FIG. 7 is a schematic representation of a further embodiment of a microfluidic radiochemical synthesis apparatus according to the present invention with an integrated microfluidic target reservoir;

FIG. 8 is a schematic representation of a further embodiment of a microfluidic radiochemical synthesis apparatus according to the present invention with a recirculating target liquid;

FIG. 9 is a schematic representation of a further embodiment of a microfluidic radiochemical synthesis apparatus according to the present invention with integrated microfluidic sensors;

FIG. 10 is a schematic representation of a further embodiment of a microfluidic radiochemical synthesis apparatus according to the present invention with an integrated HPLC column;

FIG. 11 is a schematic representation of a further embodiment of a microfluidic radiochemical synthesis apparatus according to the present invention with an integrated electrokinetic separation device;

FIG. 12 is a schematic representation of a further embodiment of a microfluidic radiochemical synthesis apparatus according to the present invention with multiple microfluidic product pathways;

FIG. 13 is a schematic representation of a further embodiment of a microfluidic radiochemical synthesis apparatus according to the present invention with microfluidic final product mixing and dispensing;

FIG. 14 is a schematic representation of a further embodiment of a microfluidic radiochemical synthesis apparatus according to the present invention with an integrated microfluidic ion exchange resin; and

FIG. 15 is a schematic representation of a further embodiment of a microfluidic radiochemical synthesis apparatus according to the present invention with an integrated microfluidic electrolytic cell.

### DETAILED DESCRIPTION OF THE PREFERRED EMBODIMENTS

The present invention will now be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

Definitions

As used herein, the singular forms “a”, “an”, “the”, include plural referents unless the context clearly dictates otherwise.

The terms “patient” and “subject” refer to any human or animal subject, particularly including all mammals.

As used herein, “radiochemical” is intended to encompass any organic or inorganic compound comprising a covalently-attached radioactive isotope (e.g., 2-deoxy-2-[¹⁸F]fluoro-D-glucose ([¹⁸F]FDG)), any inorganic radioactive ionic solution (e.g., Na[¹⁸F]F ionic solution), or any radioactive gas (e.g., [¹¹C]CO₂), particularly including radioactive molecular imaging probes intended for administration to a patient (e.g., by inhalation, ingestion or intravenous injection) for tissue imaging purposes, which are also referred to in the art as radiopharmaceuticals, radiotracers, or radioligands.

As used herein, the term “radioactive isotope” refers to isotopes exhibiting radioactive decay (i.e., emitting positrons). Such isotopes are also referred to in the art as radioisotopes or radionuclides. Radioactive isotopes are named herein using various commonly used combinations of the name or symbol of the element and its mass number (e.g., ¹⁸F, F-18, or fluorine-18). Exemplary radioactive isotopes include I-124, F-18 fluoride, C-11, N-13, and O-15, which have half-lives of 4.2 days, 110 minutes, 20 minutes, 10 minutes, and 2 minutes, respectively. The radioactive isotope is preferably dissolved in an organic solvent, such as a polar aprotic solvent where appropriate.

The term “reactive precursor” refers to an organic or inorganic non-radioactive molecule that is reacted with the radioactive isotope, typically by nucleophilic substitution, electrophilic substitution, or ionic exchange, to form the radiochemical. The chemical nature of the reactive precursor depends upon the physiological process to be studied. Typically, the reactive precursor is used to produce a radioactive labeled compound that selectively labels target sites in the body, including the brain, meaning the compound can be reactive with target sites in the subject and, where necessary, capable of transport across the blood-brain barrier. Exemplary organic reactive precursors include sugars, amino acids, proteins, nucleosides, nucleotides, small molecule pharmaceuticals, and derivatives thereof. Particularly preferred organic precursors include 1,3,4,6-tetra-O-acetyl-2-O-trifluoromethanesulfonyl-β-D-mannopyranose, a common precursor used to form [¹⁸F] FDG.

In addition to mannose triflate for FDG, there are other precursors used for producing labeled molecular probes using [¹⁸F] fluoride ion:
N²-(p-anisyldiphenylmethyl)-9-[(4-p-toluenesulfonyloxy)-3-(p-anisyldiphenylmethoxymethyl)butyl]guanine, the precursor for [¹⁸F]FHBG
N²-(p-anisyldiphenylmethyl)-9-[[1-(p-anisyldiphenylmethoxy)-3-(p-toluenesulfonyloxy)-2-propoxy]methyl]guanine, the precursor for [¹⁸F]FHPG
8-[4-(4-fluorophenyl)-4,4-(ethylenedioxy)butyl]-3-[2′-(2,4,6-trimethylphenylsulfonyloxyethyl)]-1-phenyl-1,3,8-triazaspiro[4.5]decan-4-one, the precursor for [¹⁸F]FESP
5′-O-Boc-2,3′-anhydrothymidine, precursor for [¹⁸F]FLT
N-Boc-5′-O-dimethoxytrityl-3′-O-(4-nitrophenylsulfonyl)-thymidine, precursor for [¹⁸F]FLT
N-[2-[4-(2-methoxyphenyl)-1-piperazinyl]ethyl]-4-nitro-N-2-pyridinyl-benzamide, precursor for p-[¹⁸F]MPPF
2-(1-{6-[(2-(p-toluenesulfonyloxy)ethyl)(methyl)amino]-2-naphthyl}ethylidine)malononitrile, precursor for [¹⁸F]FDDNP
1,2-bis(tosyloxy)ethane and N,N-dimethylethanolamine, precursor for [¹⁸F]fluoroethylcholine
Ditosylmethane (or dibromomethane) and N,N-dimethylethanolamine, precursor for [¹⁸F]fluorocholine

The terms “microfluidic environment” or “micro reactor” refer to a micro-scale device comprising one or more microfluidic channels or tubes (referred to as microchannels or capillaries herein) having at least one cross-sectional dimension (e.g., height, width, depth, diameter) from about 1 to about 1,000 μm, preferably from about 1 to about 500 μm, more preferably about 10 to about 500 μm. The microchannels make it possible to manipulate extremely small volumes of liquid on the order of fL to μL. The micro reactors may also comprise one or more reservoirs in fluid communication with one or more of the microchannels, each reservoir typically having a volume of about 50 to about 1,000 μL.

“Alkyl” refers to a hydrocarbon chain, typically ranging from about 1 to 20 atoms in length. Such hydrocarbon chains are preferably, but not necessarily, saturated and may be branched or straight chain, although typically straight chain is preferred. Exemplary alkyl groups include ethyl, propyl, butyl, pentyl, 1-methylbutyl, 1-ethylpropyl, 3-methylpentyl, and the like. As used herein, “alkyl” includes cycloalkyl when three or more carbon atoms are referenced.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8.

“Non-interfering substituents” are those groups that, when present in a molecule, are typically non-reactive with other functional groups contained within the molecule.

The term “substituted” as in, for example, “substituted alkyl,” refers to a moiety (e.g., an alkyl group) substituted with one or more non-interfering substituents, such as, but not limited to: C₃-C₈ cycloalkyl, e.g., cyclopropyl, cyclobutyl, and the like; halo, e.g., fluoro, chloro, bromo, and iodo; cyano; alkoxy, lower phenyl (e.g., 0-2 substituted phenyl); substituted phenyl; and the like. “Substituted aryl” is aryl having one or more non-interfering groups as a substituent. For substitutions on a phenyl ring, the substituents may be in any orientation (i.e., ortho, meta, or para).

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

“Heteroaryl” is an aryl group containing from one to four heteroatoms, preferably N, O, or S, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom which is not a carbon. Preferred heteroatoms include sulfur, oxygen, and nitrogen.

The terms “protected” or “protecting group” refers to the presence of a moiety (i.e., the protecting group) that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule, if any.

Microfluidic Apparatus and Method

The present invention provides a microfluidics-based method of synthesizing radiochemicals. The flexible, easily shielded systems provided by the invention offer the possibility of improved reactivity, yields and purity along with reduced use of reagents, the opportunity to integrate a variety of sensors, detectors, and on-line purification, and ease of control through solid-state methods.

The undesirable stable isotopes are introduced into the reaction environment by the various chemical reagents and solvents used in the synthesis process. Since the use of a microfluidic reaction zone would greatly reduce the amount of reagent and/or solvent being used, dilution of the radioactive isotope with stable isotopes will be reduced. The reduction in stable isotope dilution is particularly beneficial for probes that are used as receptor radioligands wherein the stable isotope carrier could result in a pharmacological effect, especially when used in small animal microPET investigations.

Activated isotope in the cyclotron target is only a very small percentage of the total volume and therefore adapts well to microfluidic proportions. In the case of F-18, by using various trapping techniques either with an anion resin or with electroplating, the fluoride ion can be separated from the bulk target water. The activated fluoride ion can then be manipulated in the microfluidic channels of the micro reactors of the invention with dramatically less carrier liquid. High concentration of the activated fluoride along with the inherently faster reaction times associated with micro reactors and the well-controlled microfluidic environment produces radio labeled compounds that have significantly higher synthetic yield than any conventional synthesis method.

In addition to the actual reactions that form the radiolabeled molecular imaging probe, other related processes can also be integrated into the microfluidic environment. In one embodiment, the microchip-based PET radiochemistry system will be able to perform all of the following operations in a microfluidic environment: isolate and purify the fluoride ion or other radioactive isotope out of the target liquid, quickly complete a high yield reaction with a chemical precursor (e.g., fluorination reaction) to form the radioactive isotope labeled molecular imaging probe, purify the probe molecule, and dispense the product in unit dose batches. Micro-scale synthesis will yield dramatically faster reactions and quality control (“QC”) processes, moving from hours to seconds, which has obvious advantages for production of PET compounds. Further, the system will be scalable to include parallel paths that simultaneously produce multiple batches of the same or different probes. In one embodiment, integrated sensors will monitor pH and utilize radiation detection to track the F-18 or other isotope through the process. On-chip chromatography can be used to perform inline QC and feedback loops will continuously optimize reagent and synthesis parameters. Robotic automation can be used to load and unload chips and tend to external system interfaces.

Although the present invention is primarily directed to synthesis of positron-emitting molecular imaging probes for use in PET imaging systems, the invention could be readily adapted for synthesis of any radioactive compound comprising a radionuclide, including radiochemicals useful in other imaging systems, such as single photon emission computed tomography (SPECT). Exemplary PET molecular imaging probes that could be produced using the present invention include, but are not limited to, 2-deoxy-2-[¹⁸F]fluoro-D-glucose([¹⁸F]FDG), 6-[¹⁸F]fluoro-L-3,4-dihydroxyphenylalanine ([¹⁸F]FDOPA), 6-[¹⁸F]fluoro-L-meta-tyrosine([¹⁸F]FMT), 9-[4-[¹⁸F]fluoro-3-(hydroxymethyl)butyl]guanine([¹⁸F]FHBG), 9-[(3-[¹⁸F]fluoro-1-hydroxy-2-propoxy)methyl]guanine([¹⁸F]FHPG), 3-(2′-[¹⁸F]fluoroethyl)spiperone([¹⁸F]FESP), 3′-deoxy-3′-[¹⁸F]fluorothymidine([¹⁸F]FLT), 4-[¹⁸F]fluoro-N-[2-[1-(2-methoxyphenyl)-1-piperazinyl]ethyl]-N-2-pyridinyl-benzamide([¹⁸F]p-MPPF), 2-(1-{6-[(2-[¹⁸F]fluoroethyl)(methyl)amino]-2-naphthyl}ethylidine)malononitrile([¹⁸F]FDDNP), 2-[¹⁸F]fluoro-α-methyltyrosine, [¹⁸F]fluoromisonidazole([¹⁸F]FMISO), 5-[¹⁸F]fluoro-2′-deoxyuridine([¹⁸F]FdUrd), [¹¹C]raclopride, [¹¹C]N-methylspiperone, [¹¹C]cocaine, [¹¹C]nomifensine, [¹¹C]deprenyl, [¹¹C]clozapine, [¹¹C]methionine, [¹¹C]choline, [¹¹C]thymidine, [¹¹C]flumazenil, [¹¹C]β-aminoisobutyric acid ([¹¹C]β-AIBA), and protected forms thereof.

As would be understood, protected forms of the above compounds are compounds comprising one or more labile protecting groups that can be readily removed under certain reaction conditions, such as hydrolysis conditions. One exemplary protected form of [¹¹⁸F]FDG is 2-deoxy-2-[¹⁸F]fluoro-1,3,4,6-tetra-O-acetyl-β-D-glucose, wherein the acetyl protecting groups are removed by hydrolysis to produce the desired [¹⁸F]FDG product.

In addition to tetraacetyl-FDG for FDG, other specific protected forms of radiochemicals produced and include: N²-(p-anisyldiphenylmethyl)-9-[(4-p-toluenesulfonyloxy)-3-([¹⁸F]fluoro)butyl]guanine, the intermediate for [¹⁸F]FHBG; N²-(p-anisyldiphenylmethyl)-9-[[1-(p-anisyldiphenylmethoxy)-3-([¹⁸F]fluoro)-2-propoxy]methyl]guanine, the intermediate for [¹⁸F]FHPG; 8-[4-(4-fluorophenyl)-4,4-(ethylenedioxy)butyl]-3-[¹⁸F]fluoro-1-phenyl-1,3,8-triazaspiro[4.5]decan-4-one, the intermediate for [¹⁸F]FESP; 5′-O-Boc-3′-deoxy-3′-[¹⁸F]fluorothymidine, intermediate for [¹⁸F]FLT; N-Boc-5′-O-dimethoxytrityl-3′-deoxy-3′-[¹⁸F]fluorothymidine, intermediate for [¹⁸F]FLT.

In one embodiment, the present invention provides a method for synthesizing a radiochemical in a liquid phase flowing reaction in laminar flow wherein the reagents are contacted and allowed to react in a microchannel of a micro reactor. Generally, the reaction comprises the reaction of a radioactive isotope in a polar aprotic solvent or in ionic media with a reactive precursor to form a positron-emitting molecular imaging probe. In some cases, the molecular imaging probe is formed in a single reaction step. Typically, however, the radionuclide is first reacted with a precursor compound followed by one or more additional reaction steps (e.g., deprotection steps).

As noted therein, ¹⁸F ions in a polar aprotic solvent can be reacted with an organic compound having the formula X—R, wherein R is alkyl, substituted alkyl, heterocycle, substituted heterocycle, aryl, substituted aryl, heteroaryl, and substituted heteroaryl, and X is a nucleophilic leaving group, such as a halogen, pseudohalogen, or a sulfonate ester, to form the structure, ¹⁸F-R.

In a preferred embodiment, the radiochemical synthesis reaction used in the invention comprises contacting and reacting two reagents: (1) a solution comprising a radioactive isotope dissolved in a polar aprotic solvent; and (2) a liquid organic reactive precursor dissolved in a polar aprotic solvent, wherein the reactive precursor is adapted for reaction with a radioactive isotope to form a radiochemical. The polar aprotic solvent used in each reagent can be the same or different, but is typically the same for each reagent. Exemplary polar aprotic solvents include acetonitrile, acetone, 1,4-dioxane, tetrahydrofuran (THF), tetramethylenesulfone (sulfolane), N-methylpyrrolidinone (NMP), dimethoxyethane (DME), dimethylacetamide (DMA), N,N-dimethylformamide (DMF), dimethylsulfoxide (DMSO), and hexamethylphosphoramide (HMPA). For solutions containing ¹⁸F, the radioactive isotope is typically in the form of a coordination compound consisting of a phase transfer catalyst and salt complex. One common ¹⁸F solution comprises Kryptofix 2.2.2 as the phase transfer catalyst and ¹⁸F in a salt complex with potassium carbonate (K₂CO₃).

In another preferred embodiment, the radiochemical synthesis reaction used in the invention comprises the additional step of deprotecting the radiochemical following reaction with the radioactive isotope. Typically, the deprotecting step is a hydrolysis reaction that involves contacting and reacting the radiochemical with a hydrolyzing agent, preferably an aqueous base solution or an aqueous acid solution. The aqueous base solution is preferably an alkali metal hydroxide (e.g., sodium hydroxide or potassium hydroxide) and the aqueous acid solution preferably consists of a hydrochloric acid.

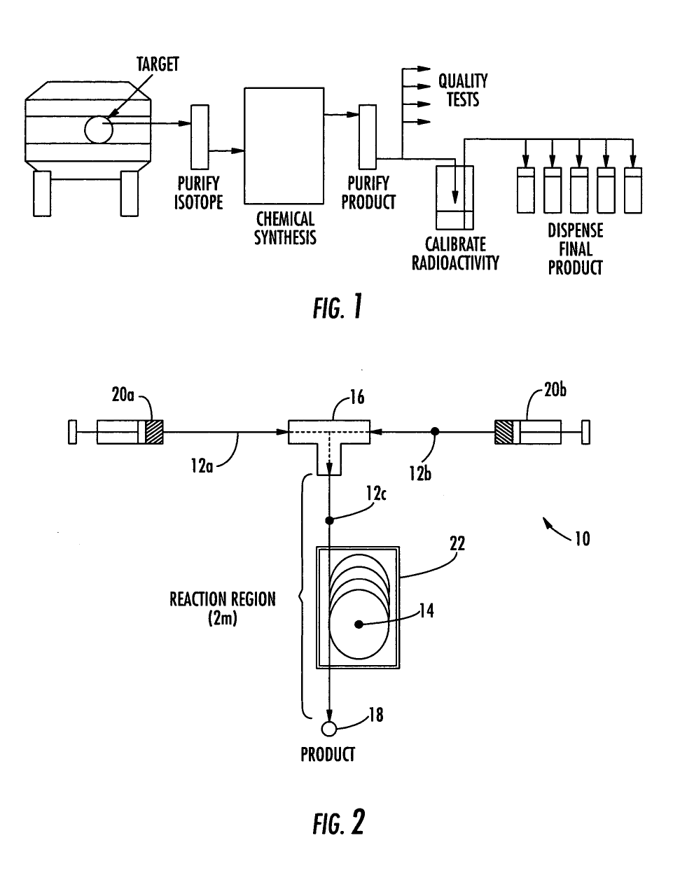

In addition to the actual reaction steps, other steps in the radiochemical production process can also be performed in a microfluidic environment. A typical radioisotope-labeled PET molecular imaging probe production process is shown in FIG. 1. As shown therein, PET radiotracers are produced using automated or manual chemistry synthesis techniques to convert raw isotope generated in a cyclotron to a useable, injectable compound. Cyclotrons accelerate ionized particles and bombard target material, such as enriched [¹⁸O] water, to produce the raw isotope. Once activated this target material is removed and purified before introduction to the synthesis process. Chemical synthesis converts the raw isotope into the desired compound and is typically followed by purification of the product. Chemical products are accurately calibrated for radioactivity and are subjected to a battery of quality control tests. Product batches are then dispensed into smaller batches or doses either manually or with automated equipment and shipped to the customer. In the process of the present invention, some or all of the above process steps are performed within a microfluidic environment.

For example, for a process utilizing fluorine-18 fluoride ion, one or more of the following steps can be performed in a microfluidic device according to the present invention:
Receive aqueous [¹⁸F] fluoride ion from the cyclotron target
Separate the [¹⁸F]fluoride ion from the water and collect the water
Generate a solution of reactive [¹⁸F] fluoride ion in an organic and/or polar aprotic solvent (acetonitrile, DMF, DMSO, etc.)
Provide a solution of a reactive precursor in an organic and/or polar aprotic solvent (acetonitrile, DMF, DMSO, etc.)
React the [¹⁸F] fluoride ion with the precursor using a S_{N}2 nucleophilic substitution reaction to create a new carbon-fluorine bond, using heat if necessary
Purify the initial [¹⁸F] fluorinated product by solid phase extraction or chromatography
React the purified initial [¹⁸F] fluorinated product with a second reagent to generate the final [¹⁸F] fluorinated product (e.g., hydrolysis of protecting group(s), if necessary)
Purify the final [¹⁸F] fluorinated product by, for example, solid phase extraction or chromatography
Desolvate the [¹⁸F] fluorinated product
Assay the purified final [¹⁸F] fluorinated product for radioactivity, UV absorbance, and conductivity/pH
Deliver the purified final [¹⁸F] fluorinated product
Dispense the purified final [¹⁸F] fluorinated product

For a process utilizing a carbon-11-labeling agent (e.g., methyl iodide, methyl triflate, carbon monoxide, hydrogen cyanide), any of the following steps can be performed within a microfluidic device according to the present invention:
Receive [¹¹C]-labeling agent from the cyclotron target or post-irradiation processor
Generate a solution of reactive [¹¹C]-labeling agent in an organic and/or polar aprotic solvent (acetonitrile, DMF, DMSO, etc.)
Provide a solution of a reactive precursor in an organic and/or polar aprotic solvent (acetonitrile, DMF, DMSO, etc.)
React the [¹¹C]-labeling agent with the precursor using a S_{N}2 nucleophilic substitution reaction or other suitable reaction to create a new carbon-nitrogen, carbon-oxygen, carbon-sulfur or carbon-carbon bond, using heat or microwave energy if necessary
Purify the initial [¹¹C]-labeled product by, for example, solid phase extraction or chromatography
React the purified initial [¹¹C]-labeled product with a second reagent to generate the final [¹¹C]-labeled product (e.g., hydrolysis of protecting group(s), if necessary)
Purify the final [¹¹C]-labeled product by solid phase extraction or chromatography
Assay the purified final [¹¹C]-labeled product for radioactivity, UV absorbance, and conductivity/pH
Desolvate the [¹¹C]-labeled product
Deliver the purified final [¹¹C]-labeled product
Dispense the purified final [¹¹C]-labeled product

A micro reactor-based radiochemical synthesis system typically comprises a micro reactor and the associated processing and control equipment required for performing the synthesis and delivering the product. In one embodiment, the radiochemistry micro reactor comprises a series or network of interconnecting microchannels that can be either cut or etched into a solid substrate (i.e., a microchip) or can comprise an assembly of glass, metal, or polymeric capillary tubing and fittings.

If a solid substrate is used, the micro reactor may comprise a microchannel network in a single layer or multiple layers of microchannels in a single chip with interconnects, if desired, connecting one layer to another. The wetted surfaces of the solid substrate and/or capillary tubing and fittings should be constructed of a material that is inert and compatible with the organic solvents and reagents used, such as glass, quartz, metal, or appropriate polymeric material (e.g., PEEK, PTFE, polystyrene, polypropylene, or acrylic polymers).

The solid substrate micro reactor may be fabricated using commercially known fabrication techniques, including but not limited to standard photolithographic procedures and wet chemical etching, with the substrate and cover plate joined using direct bonding in glass substrates and embossing in polymeric substrates.

The microchannels are in fluid communication with reservoirs for the various reagents, precursors and solvents that may be housed within the micro reactor or located remote from the micro reactor. The microchannels are also in fluid communication with reservoirs for the product(s) and for waste materials. Using the microchannels, the reagents and solvents can be brought together in a specific way and allowed to react in a controlled region of the microchannel network. Multiple ports and reservoirs may be employed as required to allow multi-step radiochemical synthesis sequences, where for example the precursor is reacted with the radioactive isotope, and then in a subsequent step (after purification if necessary), protecting groups are removed to yield the desired product.

The reagents and solvents can be moved through the microchannel network using any fluid propulsion method known in the art of microfluidics, such as electrokinetic methods (electroosmotic and electrophoretic) and/or hydrodynamic pumping. For electrokinetic pumped systems, electrodes are placed in appropriate positions such that specific voltages are delivered under microprocessor control. These voltages cause the reactants and products to move and be separated in the channels.

Hydrodynamic pumping uses appropriate external and/or internal pumps, tubing, fittings and valves to move the reactants and products through the channels by applying a positive pressure to one or more of the inlet ports of the micro reactor. Valves of any type known in the art of microfluidics, such as rotary switching valves, etched cantilever beams, bubble actuated, and inertial valves, can be placed at the microchannel junctions to direct flow. Laminar flow with a planar velocity profile characterizes the principles of operation inside the microchannels and can be utilized to control diffusion and reaction properties.

Monitoring of the reactants and products may be accomplished using various sensors and detectors that can be integrated into the micro reactor. For example, pH sensors, conductivity sensors, radiation sensors, and liquid and gas chromatography devices can be integrated into the microfluidic apparatus. Alternatively, the sensors and detectors can be used remotely from the micro reactor for analysis and testing.

### EXEMPLARY EMBODIMENTS

A number of exemplary embodiments are described below. These embodiments are provided for illustrative purposes only and should not be construed as limiting the invention. For example, it would be understood that microchips comprising additional ports, reservoirs or microchannels not shown in the exemplary structures described below could be readily utilized in the present invention.

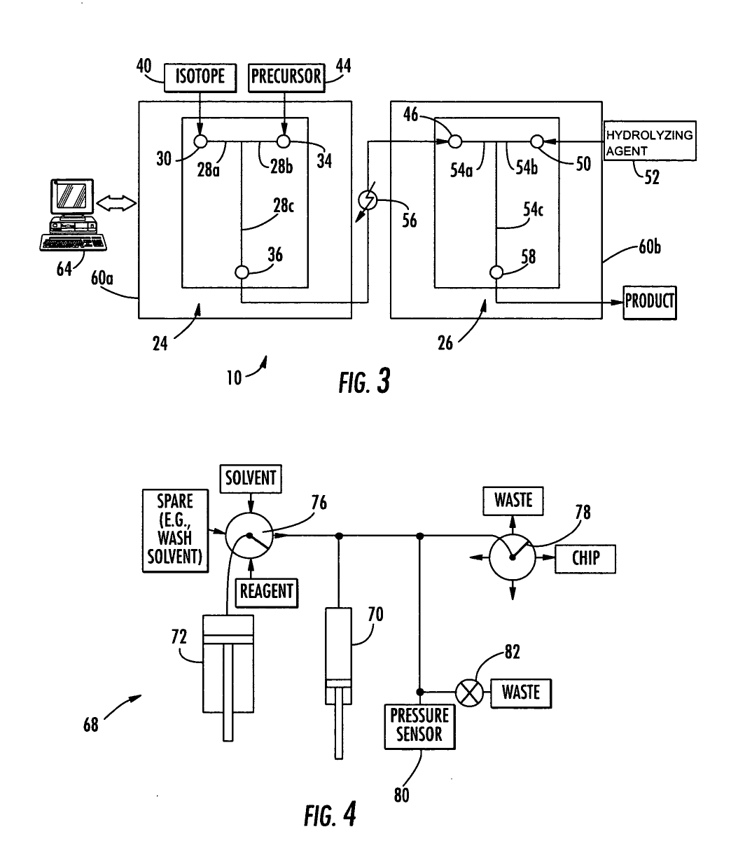

In a version of a micro reactor 10 of the invention shown in FIG. 2, the microchannels, 12a, 12b, and 12c, are formed by connecting three lengths of capillary tubing to a T-shaped member 16. The reactants are introduced through ports or reservoirs at each end of the channels, 12a and 12b, forming the cross of the “T” and are brought together through the “T-junction” to react in the third channel 12c. The product is delivered to a reservoir 18 at the end of the reaction channel 12c. A portion 14 of the reaction channel 12c can be heated by a heating source 22 to promote the desired reaction. Pumps such as syringe pumps, 20a and 20b, are used to propel the reagents through the micro reactor 10. Any heating unit can be used as heating source 22, including but not limited to resistive heating, localized and non-localized microwave heating and Peltier devices. Exemplary pumps for use in the invention include but are not limited to syringe pumps such as a Harvard PHD 2000. An embodiment of the device shown in FIG. 2 was used in Examples 1 and 2.

FIG. 3 illustrates a further embodiment of a micro reactor 10 comprising a first microchip 24 and a second microchip 26. The first microchip 24 is designed to react a radioactive isotope with a reactive precursor and the second microchip 26 is designed to deprotect the radiochemical product of the first microchip. The first microchip 24 comprises an interconnecting microchannel network comprising a first microchannel segment 28a in fluid communication with a first inlet 30 of the microchip, a second microchannel segment 28b in fluid communication with a second inlet 34 of the microchip, and a third microchannel segment 28c in fluid communication with the outlet 36 of the microchip. As shown, all three microchannel segments intersect within the microchip 24. The first inlet 30 of the first microchip 24 is in fluid communication with a supply 40 of a radioactive isotope, such as a solution of ¹⁸F fluoride. As noted above, the supply 40 of radioactive isotope is preferably a solution of radioactive isotope dissolved in a polar aprotic solvent. The second inlet 34 of the first microchip 24 is in fluid communication with a supply 44 of a reactive precursor, such as a supply of a liquid organic precursor dissolved in a polar aprotic solvent as described above.

The outlet 36 of the first microchip 24 is in fluid communication with a first inlet 46 of the second microchip 26. Preferably, capillary tubing having an inner diameter of no more than 1 mm is used to connect the two microchips. As shown, it is preferred for the effluent from the first microchip 24 to pass through a heat exchanger 56 to reduce the temperature of the effluent prior to introducing the effluent into the second microchip 26. The heat exchanger can be any known type of heat exchanger, such as a water bath or other liquid maintained at a known temperature. The second inlet 50 of the second microchip 26 is in fluid communication with a supply 52 of an aqueous base solution. The microchannel network of the second microchip 26 includes a first microchannel segment 54a in fluid communication with a first inlet 46 of the microchip, a second microchannel segment 54b in fluid communication with a second inlet 50 of the microchip, and a third microchannel segment 54c in fluid communication with the outlet 58 of the microchip. As shown, all three microchannel segments intersect within the microchip 26.

Both microchips are in contact with a heat source, 60a and 60b, capable of heating each microchip independently. Suitable heat source include but are not limited to resistive heating, localized and non-localized microwave heating and Peltier devices. As would be understood, various sensors (e.g., flow sensors, radioactivity sensors, pressure sensors, temperature sensors, and the like) and other apparatus components (e.g., valves, switches, etc.) (not shown) can be integrated into the micro reactor 10 and connected to a computer 64 for process control and monitoring purposes. Syringe pumping systems or other pumping devices (not shown), such as the syringe pumping system described below in connection with FIG. 4, can be incorporated into the micro reactor 10 in order to propel the reagents through the microchannels. Preferably, the reagents flow through each microchip in laminar flow and at a flow rate of about 1 to about 120 μL/min.

In operation, radioactive isotope will flow into the first microchip 24 from the isotope supply 40 and reactive precursor will flow into the first microchip from precursor supply 44. The two reactants will contact each other and react in a microchannel 28c of the microchip 24. The heat source 60a maintains the microchannel network at the desired reaction temperature, which is preferably at least about 85° C., more preferably at least about 95° C.

In one embodiment, the temperature of the microchannel network of the first microchip 24 is maintained at a temperature of about 60 to about 100° C., preferably 85 to 100° C. The preferred reaction temperature for optimal yield is above the boiling point (at 1 atm) of certain preferred polar aprotic solvents, such as acetonitrile. As a result, it is preferred to maintain the pressure within the microchannel network of the first microchip 24 at a level sufficient to maintain the solvent in liquid form at the desired reaction temperature. In one embodiment, the pressure in the first microchip 24 is at least about 2 bar, more preferably at least about 4 bar. Preferably, the pressure in the first microchip 24 is between about 2 and about 400 bar. The pressure in the first microchip 24 can be elevated to the desired level by, for example, connecting capillary tubing having a smaller inner diameter than the microchannel network of the first microchip to the outlet 36 of the first microchip.

The effluent from the first microchip 24 passes through a heat exchanger 56 that reduces the temperature of the effluent, preferably to a temperature of about 0 to about 30° C. In one embodiment, the heat exchanger is a water bath having a temperature of about 0 to about 30° C., the capillary tubing carrying the effluent from microchip 24 being immersed in the water bath. Thereafter, the cooled effluent from the first microchip 24 in introduced into the second microchip 26 along with base from base supply 52. The second microchip 26 is maintained at a desired temperature using the associated heat source 60b. Preferably, the microchannel network of the second microchip 26 is maintained at a temperature of about 0 to about 35° C., more preferably about 20 to about 35° C. The radiochemical in the effluent stream from the first microchip 24 contacts the base and reacts with the base to remove protecting groups from the radiochemical by hydrolysis. For example, in the synthesis of [¹⁸F]FDG, the effluent stream from the first microchip 24 may contain 2-deoxy-2-[¹⁸F]fluoro-1,3,4,6-tetra-O-acetyl-β-D-glucose, wherein the acetyl protecting groups are removed by reaction with the aqueous base solution (i.e., by hydrolysis) to form the final desired product. The product stream is then collected from outlet 58 of the second microchip 26.

FIG. 4 illustrates an embodiment of one a preferred syringe pumping system 68 that can be used with the present invention. As noted above, a syringe pumping system or other pumping apparatus can be utilized to propel each reagent through the microchannels of the micro reactor 10. In one embodiment, a syringe pumping device is used to pump each reagent through the micro reactor 10, meaning a syringe pumping system is provided for the reactive precursor, the isotope-containing solution, the base solution, and any other solutions adapted for pumping through the micro reactor, such as wash solvents and the like. Preferably, each of the reagents (e.g., isotope, reactive precursor, and base solution) is pumped through the micro reactor 10 using a separate syringe pumping apparatus. As shown in FIG. 4, a preferred syringe pumping system 68 comprises a first syringe 70 and a second syringe 72, wherein the second syringe is of sufficient size to aspirate a volume twice the volume of the first syringe. The two syringes, 70 and 72, are in fluid communication with each other such that the two syringes are capable of providing continuous flow by sequentially aspirating and dispensing.

As shown, a first valve 76 is in fluid communication with the second larger syringe 72 so that the source from which the second syringe aspirates can be switched as desired. A second valve 78 is operatively positioned downstream from the first valve 76 so as to control the destination of the material being pumped. In this manner, the second valve 78 is used to direct the material being pumped to, for example, the micro reactor or a waste port. A pressure sensor 80 is preferably placed in fluid communication with the two syringes, 70 and 72. As shown, the pressure sensor can be placed in a line leading to a waste port 82.

In operation, as the second larger syringe 72 dispenses, the first syringe 70 aspirates half of the volume dispensed by the second syringe. Once the second syringe 72 has completed dispensing, the first syringe 70 begins dispensing and the second syringe begins to aspirate from the desired source, which can be controlled by manipulating the first valve 76. This cycle continues to achieve continuous flow through the microfluidic environment.

FIG. 5 illustrates a micro reactor 10 embodiment wherein the reservoirs, 86a, 86b, and 86c, of the reagents used in the radiochemical synthesis process are located in the microfluidic environment (i.e., on the microchip), thereby further exploiting the advantages of manipulating fluids at the micro scale. The integration of reagent reservoirs on the microchip will greatly reduce the volume of reagents consumed due to less dead volume, simplify design, and increase reliability of the system. A single chip could be a self-contained disposable or reusable device that has everything required for synthesis of a compound and thus replacing the much larger and more complex synthesis instruments that are current state of the art.

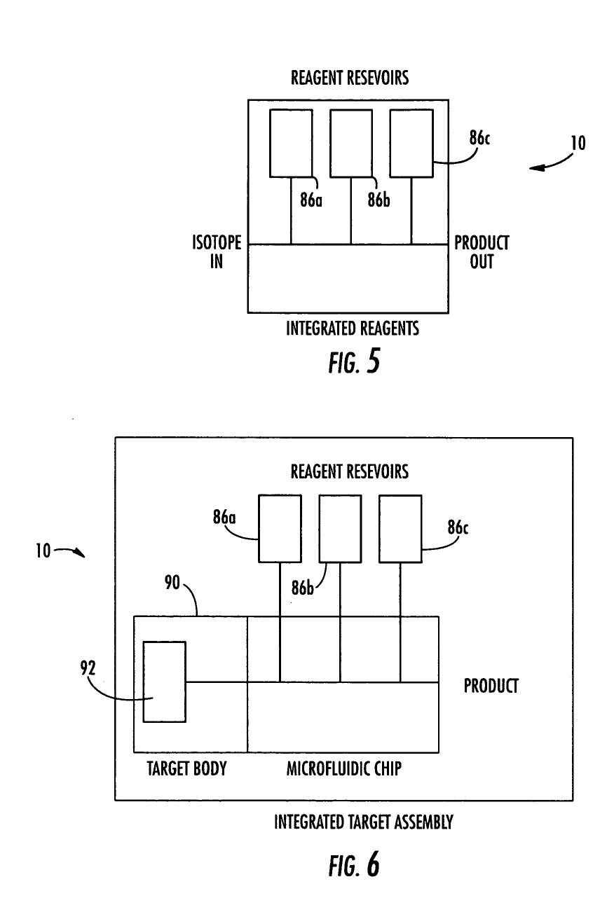

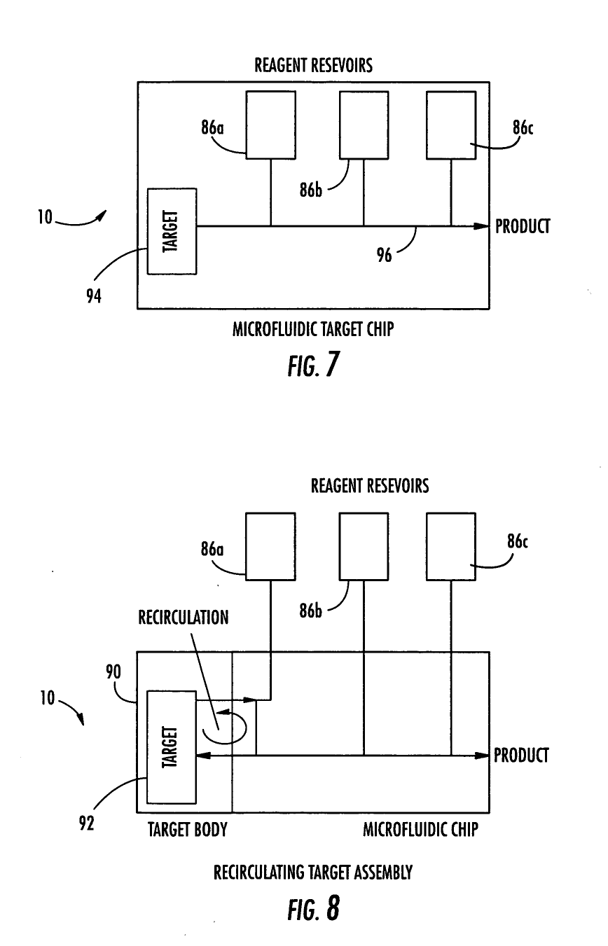

FIG. 6 illustrates a micro reactor 10 embodiment integrated with the target body assembly 90 where the radioisotope is collected. Current state of the art PET radiochemical synthesis requires bombardment of target material in a cyclotron, then unloading the target to automated or manual chemistry synthesis instruments. Volumes are typically 1 to 5 ml and transport distances can be up to 100 feet. By integrating microfluidic channels, reservoirs, devices, and reactors, many chemical processes can be performed local to the target. FIG. 6 illustrates an embodiment where reagents are stored in reservoirs, 86a, 86b, and 86c, on the same microfluidic chip that is integrated with the target assembly 90 and proximal to the metal target 92 loaded with target material. This allows immediate local synthesis, reducing time, risk of contamination, radiation exposure, and considerably reduces cost. Further integration is shown in FIG. 7, which illustrates a micro reactor 10 wherein a target chamber 94 and a plurality of reagent chambers, 86a, 86b, and 86c, are etched into a single microfluidic chip along with the interconnecting microchannel network 96. This embodiment of the micro reactor 10 should be constructed of a thermally conductive, chemically resistant material.

FIG. 8 is a further micro reactor 10 embodiment that integrates the metal cyclotron target 90 with the microfluidic device in a bonded or coupled assembly. In this embodiment, the target material is passed from the metal target 92 to the adjoining microfluidic chip and processed in a recirculating continuous flow pattern proximal to the micro-reactor where the activated isotope is removed and the unactivated target material returns to the target for irradiation. The activated isotope is further processed inside the microfluidic chip to produce the positron-emitting molecular imaging probe. In this manner, the target material is continuously bombarded in a cyclotron while being circulated out of the beam strike area to allow the activated isotope to be trapped, then recirculated back into the beam strike area. Thus, radioisotopes can be continuously processed in real-time as needed.

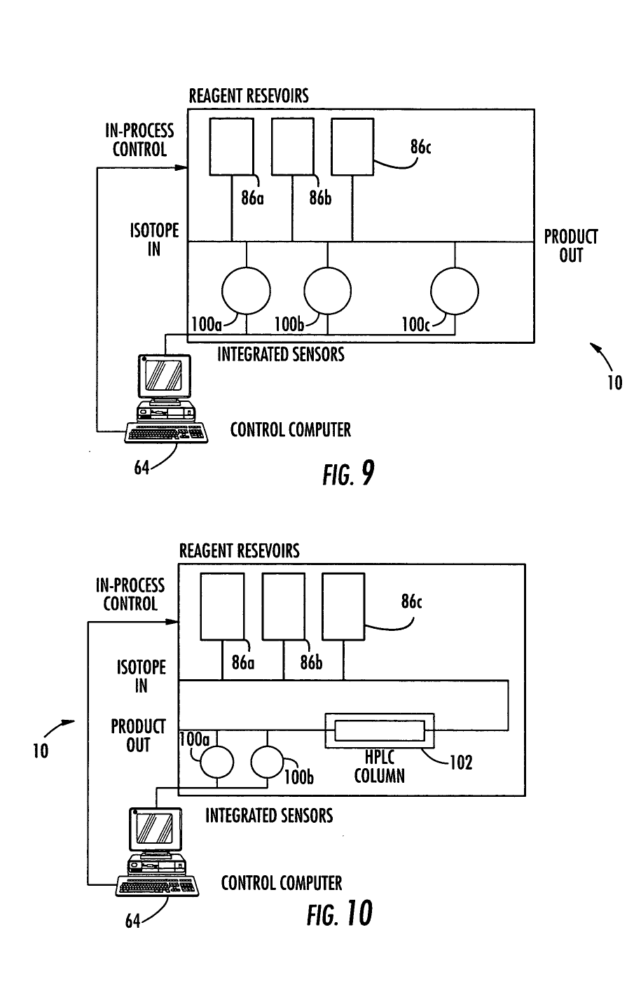

FIG. 9 illustrates a micro reactor 10 embodiment including sensors, 100a, 100b, and 100c, integrated into the microfluidic structure. The use of integrated microfluidic sensors/detectors, such as pH sensors, conductivity sensors, radiation sensors, liquid and gas chromatography devices, and mass spectroscopy devices, will allow in-process measurements of starting materials, intermediate materials, and final products generated in the microfluidic circuit. A computer 64 comprising control software can utilize these in-process measurements to adjust flow or reaction parameters and test for clogs, leaks, or reaction failures in real-time and then make decisions on how to correct any deviations in the continuous flow process of the microfluidic circuit. Current technology operating at the macroscale utilizes in-process sensing of radiation, temperature, and pressure, but has no automated capability to correct the batch mode processes.

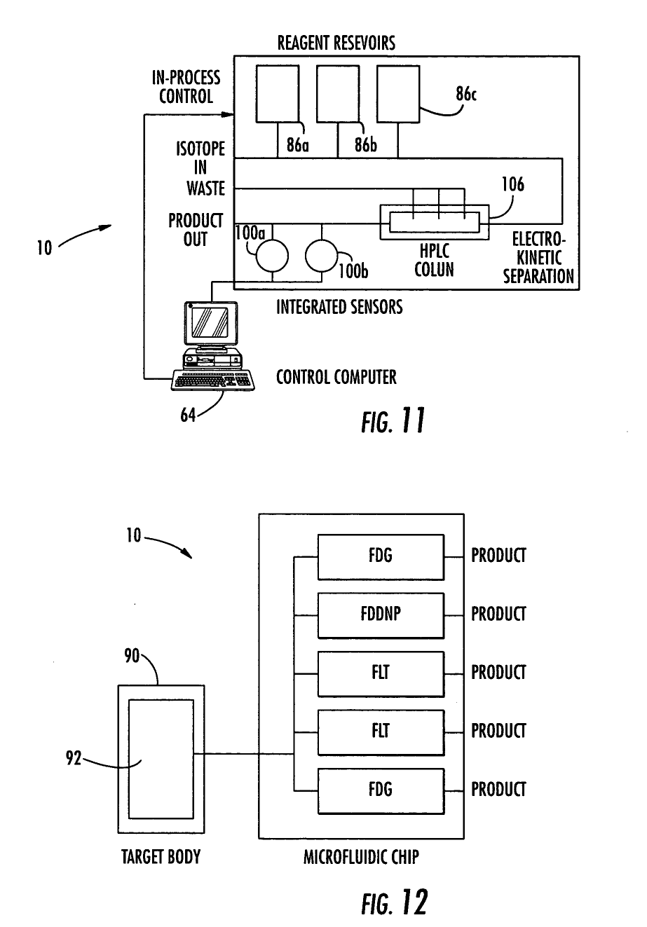

Current state of the art production techniques require PET radiolabeled products to be purified following synthesis to be useful injectable compounds. Current purification techniques include HPLC separation and or solid phase extraction to remove unwanted elements and to purify the final product. In one embodiment of the present invention shown in FIG. 10, such purification processes are also integrated into the micro reactor 10 device. Incorporation of both solid phase resins and in-line HPLC column 102 onto the microfluidic chip will allow continuous flow product purification in a much smaller volume with greatly improved reliability. In addition to these techniques, FIG. 11 illustrates the use of electrokinetic flow as an additional means to separate constituents and to extract the purified final product. In this embodiment, electric fields are applied to separate constituents by capillary electrophoresis and electrochromatography using an electrokinetic separation device 106. Further, by utilizing the electric potential and viscous drag differences of unlike molecules, constituents can be separated and concentrated in a microfluidic channel by driving electrokinetically in one direction, and hydraulically in the opposite direction. Once separated and concentrated, the constituents can be directed into channels for dispensing or further separation.

One of the key strengths in microfluidic design is the ability to parallel process solutions with high accuracy and minimal loss. To leverage this capability, one embodiment of the present invention, shown in FIG. 12, the microfluidic device 10 is configured to produce multiple PET radiotracers or multiple paths of the same tracer in parallel. The radioactive isotope would be transferred from the cyclotron to the microfluidic chip, then separated and processed in parallel as needed. Redundancy gives the system improved reliability and capability to automatically correct problems detected during synthesis. FIG. 12 illustrates five parallel circuits for five different nucleophilic processes. This concept can be applied to electrophilic and gas processing as well as multiple channels of the same process.

The micro reactor 10 embodiment of FIG. 13 includes integration of radiation measurement and accurate volume control, which allows on-chip quantification of activity per unit volume and the automatic dispensing of calibrated dose volumes. An inline sensor 108 measures radioactivity as the liquid moves through the chip or is accumulated in an on-chip chamber. For instance, beta radiation can be measured by integrating a semiconductor layer with etched photo diodes in the microfluidic chip that is in close proximity to the microchannel. Gamma radiation can be measured using scintillating detectors in single photon and coincidence photon collection configurations. Computer control dispenses the desired amount of activity into product containers 110 and also adds saline to deliver the desired volume.

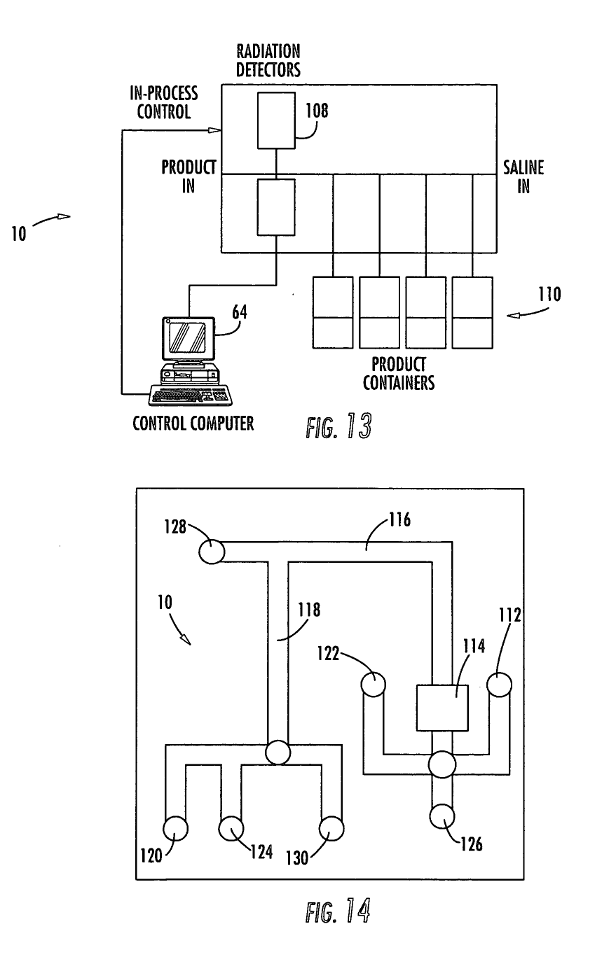

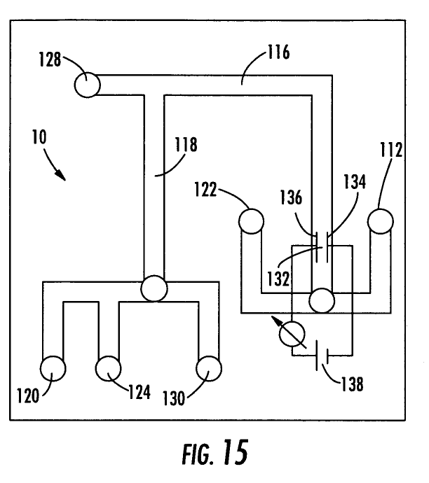

In yet another embodiment of the present invention, the radioactive isotope is separated from the target liquid via a separation device integrated into the microfluidic device, as shown in FIGS. 14 and 15. An exemplary device including an ion exchange resin as the radioisotope separation device is shown in FIG. 14. As shown, micro reactor 10 comprises a port 112 wherein the radioactive isotope in the target liquid is introduced into the device and allowed to flow across ion exchange resin 114 and into microchannel 116. The radioactive isotope remains ionically bound to resin 114 while the liquid flows through microchannels 116 and 118 to waste target liquid port 120. A polar aprotic solvent is introduced into the microchip 10 through a port 122. The polar aprotic solvent flows through microchannels 116 and 118 to collection port 124. This step is essential as it serves to clean the microchannels of microchip 10 before the organic precursor and the radioactive isotope are allowed to come in contact. An eluent dissolved in a polar aprotic solvent is introduced into the microchip 10 through port 126 and the radioactive isotope is ionically exchanged for the counter ion in the eluent as it passes through resin 114, thus releasing the isotope into the polar aprotic solvent. The organic or inorganic precursor is then introduced to the microchip 10 through port 128. The polar aprotic solvent containing the isotope and the precursor meet at the junction of microchannels 116 and 118. The two reactants react to form the positron-emitting molecular imaging probe in microchannel 118 and the product is collected in product port 130.

FIG. 15 illustrates an embodiment of microchip 10 wherein the isotope separation device is an electrolytic cell. As shown, microchip 10 comprises a port 112 wherein the radioactive isotope in the target liquid is introduced into the device and allowed to flow across electrolytic cell 132, which comprises an anode 134 and a cathode 136, and into microchannel 116 while a voltage is applied to the electrolytic cell by a DC power supply 138. The radioactive isotope remains on the anode 134 of the electrolytic cell 132 while the target liquid flows through microchannels 116 and 118 to target liquid port 120. The voltage across the electrolytic cell 132 is maintained while a polar aprotic solvent flows from port 122 through microchannels 116 and 118 to collection port 124. Polar aprotic solvent is again introduced through port 122 and the voltage from power supply 138 is reversed, thereby releasing the isotope into the polar aprotic solvent. The organic precursor is then introduced to the microchip 10 through port 128. The polar aprotic solvent containing the isotope and the precursor meet at the junction of microchannels 116 and 118. The two reactants react to form the positron-emitting molecular imaging probe in microchannel 118 and the product is collected in product port 130.

The anion exchange resin or electrochemical cell shown in FIGS. 14 and 15 could be integrated on the microchip or could be a separate unit that interfaces with the microchip. Multiple anion exchange resin modules or multiple electrochemical cells could be present on a single chip allowing multiple syntheses to take place on the same chip unit.

The following examples are given to illustrate the invention, but should not be considered in limitation of the invention. Unless otherwise indicated, all conversion data was obtained by collecting a sample and spotting 1-2 μL of the sample onto a Whatman aluminum backed SIL G TLC plate. The plate was then developed in a TLC chamber using a 95%/5% acetonitrile/water (v/v) mixture as the mobile phase. After development, the plate was scanned using a Bioscan AR 2000 radio-TLC scanner. Unless otherwise noted, each ¹⁸F solution used in the experiments comprises Kryptofix 2.2.2/K₂CO₃/¹⁸F-dissolved in acetonitrile. Mannose triflate referred to in the examples is also known as 1,3,4,6-tetra-O-acetyl-2-O-trifluoromethanesulfonyl-β-D-mannopyranose. Measurements of pH were made using Universal Indicator solution.

### EXAMPLE 1

### Radiochemical Synthesis of [¹⁸F]fluoroethyl tosylate

An embodiment of the micro reactor of the invention, which is shown in FIG. 2, was constructed using fused silica capillary tubing (360 μm OD×100 μm ID) and Microtight® fittings (Upchurch Scientific). Two pieces of capillary tubing exactly 25 cm long were attached to the opposite sides of a MicroTee (Part No. P-775, Upchurch Scientific, 150 μm thru-holes, 29 nL swept volume) and a third piece of capillary tubing 2 m long was attached to the remaining orthogonal position on the MicroTee. The chemical and radiochemical reagents were introduced into and moved through the reactor using a syringe pump (Harvard PHD 2000) and two 1 mL polypropylene syringes. A central 125 cm portion of the 2 m reaction channel was formed into four 10 cm diameter loops that were secured together. This section of four loops was placed in a water bath that was heated to 65-70° C. The output end of the reaction channel was placed into a small test tube that contained 700 μL of acetonitrile.

Ethylene glycol di-tosylate (8.4 mg, 22.7 μmol) was dissolved in 200 μL acetonitrile, and about 140 μL of this solution (containing 15.9 μmol) was loaded into one of the 1 mL syringes. Dry [¹⁸F] fluoride ion in acetonitrile was prepared by the standard method: [¹⁸O] water was irradiated with 11 MeV protons. At the end of bombardment the [¹⁸O] water was transferred through a small anion exchange resin (MP-1) column to trap the [¹⁸F] fluoride ion. The [¹⁸F] fluoride ion was then released from the resin column using 0.6 mL of potassium carbonate (2.8 mg) in water, and delivered into a vessel containing a solution of Kryptofix 222 (1.0 g) in acetonitrile (1 mL).

The acetonitrile was evaporated and three additional portions of acetonitrile (0.6 mL) were added and evaporated. After cooling, acetonitrile (250 μL) was added to the dry [¹⁸F] fluoride ion residue, mixed by bubbling with argon, and 140 μL of this solution was transferred to the other 1 mL syringe. This solution contained about 260 mCi of [¹⁸F] fluoride ion. Once the two syringes were loaded with equal volumes of reagent solution, the syringe pump was started at a flow rate of 4 μL/min. After 1 minute the flow rate was changed to 1.0 μL/min. The two solutions were pumped through the 2 m reaction channel that included the 125 cm portion heated to 65-70° C. At 1 μL/min, the reagents had a residence time of 5 minutes in the heated reaction zone. After about 100 minutes, the collected product solution was diluted with acetonitrile to make the total volume equal to 1 mL. The product reaction mixture was injected onto a semi-prep HPLC column (Phenomenex Luna, 5μ C18, 250×10 mm, mobile phase acetonitrile/water, 50:50, 4 mL/min), and the eluent monitored using UV at 254 nm and a flow-through radioactivity detector. The unreacted [¹⁸F] fluoride ion eluted at about 3 minutes, and the desired [¹⁸F] fluoroethyl tosylate eluted at 13-15 minutes.

### EXAMPLE 2

### Radiochemical synthesis of 2-deoxy-2-[¹⁸F]fluoro-1,3,4,6-tetra-O-acetyl-β-D-glucose

Using the same micro reactor apparatus described in Example 1 above, a solution of mannose triflate (4.4 mg, 9.2 μmol)) in acetonitrile (140 μL) was loaded into a 1 μL syringe. An anhydrous solution of [¹⁸F] fluoride ion (210 mCi) in 140 μL of acetonitrile (prepared as described in Example 1 above) was transferred to a second 1 μL syringe. Once the two syringes were loaded with equal volumes of reagent solution, the syringe pump was started at a flow rate of 4 μL/min. After 1 minute the flow rate was changed to 1.0 μL/min. The two solutions were pumped through the 2 m reaction channel that included the 125 cm portion heated to 65-70° C. over a period of 100 minutes. After about 100 minutes, the collected product solution was analyzed by radioTLC (silica gel, ether). In addition to unreacted [¹⁸F] fluoride ion at R_{f}=0.0, the desired radiofluorinated product was detected at R_{f}=0.65.

### EXAMPLE 3

### Radiochemical synthesis of 2-deoxy-2-[¹⁸F]fluoro-1,3,4,6-tetra-O-acetyl-β-D-glucose

[¹⁸F] fluoride ion in acetonitrile was prepared by the following method: [¹⁸O] water was irradiated with 11 MeV protons. At the end of bombardment the [¹⁸O] water was transferred through a Waters QMA Light anion exchange cartridge to trap the [¹⁸F] fluoride ion. The [¹⁸F] fluoride ion was then released from the resin column using 1.0 mL of potassium carbonate (5.5 mg) in a solution of 97.5% acetonitrile/2.5% water by weight. This mixture was delivered in to a 20 mL glass vial where an additional 9 mL of dry acetonitrile was added. This resulted in a [¹⁸F] fluoride solution containing 0.25% water in acetonitrile by weight.

A micro reactor system was constructed using a microchip having a T-shaped microchannel with two inlet ports and an outlet port. Using a Hamilton Company, having an address of 4970 Energy Way, Reno, Nev. 89502, syringe system comprising SGE gas tight syringe needles, a solution of mannose triflate and a [¹⁸F] fluoride solution, prepared as described above in this example, were pumped separately into an inlet of the microchip. The outlet was connected to a 2 m length of fused silica capillary, 100 μm×360 μm, of which 1.4 m was placed into an oil bath allowing heating of the reaction zone. The system was allowed to equilibrate for 15 minutes at a flow rate of 5 μL/min and the product was collected for a period of 3 minutes into a HPLC vial for analysis by TLC. Highest yield observed: 63%.

### EXAMPLE 4

### Radiochemical synthesis of 2-deoxy-2-[¹⁸F]fluoro-1,3,4,6-tetra-O-acetyl-β-D-glucose

The micro reactor system of Example 3 was used, except the oil bath was placed in a water bath to improve temperature control and stability and held at a temperature of 95° C. The [¹⁸F] fluoride solution was prepared in the same manner as in Example 3. A solution of mannose triflate and an isotope containing solution consisting of fluorine-18 fluoride containing 0.25% water by volume were pumped separately into an inlet of the microchip. The system was allowed to equilibrate for 5 minutes at a flow rate of 5 μL/min and the product was sampled straight from the capillary onto the TLC plate. Highest yield observed: 91%.

### EXAMPLE 5

### Radiochemical synthesis of 2-deoxy-2-[¹⁸F]fluoro-1,3,4,6-tetra-O-acetyl-β-D-glucose

The micro reactor system of Example 4 was used, except a second fused silica capillary section was connected to the outlet, the second capillary section being 2m in length, 75 μm×360 μm, which increased the back pressure by 2.6 Bar. The second outlet capillary section was placed in a cooled water/ice bath. The [¹⁸F] fluoride solution was prepared in the same manner as in Example 3. The syringes were set at 10 μL/min and the product was collected for 3 minutes into a HPLC vial for analysis by TLC. Average yield: 91.0%.

### EXAMPLE 6

### Radiochemical synthesis of 2-deoxy-2-[¹⁸F]fluoro-1,3,4,6-tetra-O-acetyl-β-D-glucose

The micro reactor system of Example 5 was used to determine effect of temperature and flow rate on yield. The [¹⁸F] fluoride solution was prepared in the same manner as in Example 3. Multiple experimental runs were conducted at varying flow rates while holding the reaction temperature constant and at varying temperature while holding the flow rate constant. Increasing yield was observed as temperature increased. Decreasing yield was observed with increasing flow rate. A constant flow rate of 20 μl/min at a reaction temperature of 98° C. resulted in an average yield of 97.7%.

1. A method for synthesizing a radiochemical in a microfluidic environment, the method comprising:
i) providing a micro reactor comprising a first inlet port, a second inlet port, an outlet port, and at least one microchannel in fluid communication with the first and second inlet ports and the outlet port;
ii) introducing a reactive precursor into the first inlet port of the micro reactor, the reactive precursor adapted for reaction with a radioactive isotope to form a radiochemical;
iii) introducing a solution comprising a radioactive isotope into the second inlet port of the micro reactor;
iv) contacting the reactive precursor with the isotope-containing solution in the microchannel of the micro reactor;
v) reacting the reactive precursor with the isotope-containing solution as the reactive precursor and isotope-containing solution flow through the microchannel of the micro reactor, said reacting step resulting in formation of a radiochemical; and
vi) collecting the radiochemical from the outlet port of the micro reactor.

2. The method of claim 1, wherein the radioactive isotope is dissolved in a polar aprotic solvent.

3. The method of claim 2, wherein the polar aprotic solvent is selected from the group consisting of acetonitrile, acetone, N,N-dimethylformamide (DMF), dimethylsulfoxide (DMSO), and hexamethylphosphoramide (HMPA).

4. The method of claim 1, wherein the radioactive isotope is selected from the group consisting of fluorine-18 fluoride, carbon-11, nitrogen-13, and oxygen-15.

5. The method of claim 1, wherein the radioactive isotope is fluorine-18 fluoride in the form of a coordination compound consisting of a phase transfer catalyst and salt complex.

6. The method of claim 1, wherein the reactive precursor is an organic molecule selected from the group consisting of sugars, amino acids, proteins, nucleosides, nucleotides, small molecule pharmaceuticals, and derivatives thereof.

7. The method of claim 1, wherein the reactive precursor is an organic molecule having the structure X—R, wherein R is selected from the group consisting of alkyl, substituted alkyl, heterocycle, substituted heterocycle, aryl, substituted aryl, heteroaryl, and substituted heteroaryl, and X is a nucleophilic leaving group.

8. The method of claim 7, wherein X is a halogen or a pseudohalogen.

9. The method of claim 1, wherein the reactive precursor is dissolved in a polar aprotic solvent.

10. The method of claim 1, wherein the reactive precursor and the isotope-containing solution are moved through the micro reactor using at least one pump.

11. The method of claim 1, further comprising heating the reactive precursor and isotope-containing solution during said reacting step.

12. The method of claim 1, wherein the micro reactor comprises a first microchannel segment in fluid communication with the first inlet of the micro reactor, a second microchannel segment in fluid communication with the second inlet of the micro reactor, and a third microchannel segment in fluid communication with the outlet of the micro reactor, wherein the first, second and third microchannel segments intersect.

13. The method of claim 1, wherein the radiochemical collected from the micro reactor is selected from the group consisting of 2-deoxy-2-[¹⁸F]fluoro-D-glucose ([¹⁸F]FDG), 6-[¹⁸F]fluoro-L-3,4-dihydroxyphenylalanine([¹⁸F]FDOPA), 6-[¹⁸F]fluoro-L-meta-tyrosine ([¹⁸F]FMT), 9-[4-[¹⁸F]fluoro-3-[¹⁸F]fluorocholine, [¹⁸F]fluoroethylcholine, 9-[4-[¹⁸F]fluoro-3-(hydroxymethyl)butyl]guanine([¹⁸F]FHBG), 9-[(3-[¹⁸F]fluoro-1-hydroxy-2-propoxy)methyl]guanine([¹⁸F]FHPG), 3-(2′-[¹⁸F]fluoroethyl)spiperone([¹⁸F]FESP), 3′-deoxy-3′-[¹⁸F]fluorothymidine([¹⁸F]FLT), 4-[¹⁸F]fluoro-N-[2-[1-(2-methoxyphenyl)-1-piperazinyl]ethyl]-N-2-pyridinyl-benzamide([¹⁸F]p-MPPF), 2-(1-{6-[(2-[¹⁸F]fluoroethyl)(methyl)amino]-2-naphthyl}ethylidine)malononitrile([¹⁸F]FDDNP), 2-[¹⁸F]fluoro-α-methyltyrosine, [¹⁸F]fluoromisonidazole([¹⁸F]FMISO), 5-[¹⁸F]fluoro-2′-deoxyuridine([¹⁸F]FdUrd), [¹¹C]raclopride, [¹¹C]N-methylspiperone, [¹¹C]cocaine, [¹¹C]nomifensine, [¹¹C]deprenyl, [¹¹C]clozapine, [¹¹C]methionine, [¹¹C]choline, [¹¹C]thymidine, [¹¹C]flumazenil, [¹¹C]β-aminoisobutyric acid ([¹¹Cβ-AIBA), and other small physiologically-active molecules that are labeled using fluoride ion and protected forms thereof.

14. The method of claim 1, further comprising performing at least one additional method step in a microfluidic environment, the at least one additional method step being selected from the group consisting of deprotecting the radiochemical, purifying the radiochemical, and assaying radioactivity of the radiochemical.

15. A method for synthesizing a fluorine-18 fluoride labeled radiochemical in a microfluidic environment, the method comprising:
i) providing a micro reactor comprising a first inlet port, a second inlet port, an outlet port, and at least one microchannel in fluid communication with the first and second inlet ports and the outlet port;
ii) introducing a liquid organic reactive precursor dissolved in a polar aprotic solvent into the first inlet port of the micro reactor, the organic reactive precursor adapted for reaction with fluorine-18 fluoride to form a radiochemical;
iii) introducing a solution comprising fluorine-18 fluoride dissolved in a polar aprotic solvent into the second inlet port of the micro reactor;
iv) contacting the organic reactive precursor with the isotope-containing solution in the microchannel of the micro reactor;
v) reacting the organic reactive precursor with the fluorine-18 fluoride solution in a nucleophilic substitution reaction as the reactive precursor and fluorine-18 fluoride solution flow through the microchannel of the micro reactor, said reacting step resulting in formation of a fluorine-18 fluoride labeled radiochemical; and
vi) collecting the fluorine-18 fluoride labeled radiochemical from the outlet port of the micro reactor.

16. The method of claim 15, wherein said reacting step is conducted at a temperature of 65-100° C.

17. The method of claim 15, wherein the polar aprotic solvent is selected from the group consisting of acetonitrile, acetone, N,N-dimethylformamide (DMF), dimethylsulfoxide (DMSO), and hexamethylphosphoramide (HMPA).

18. The method of claim 15, wherein the radioactive isotope is fluorine-18 fluoride in the form of a coordination compound consisting of a phase transfer catalyst and salt complex.

19. The method of claim 15, wherein the said reacting step is conducted where the water content, by weight, of the [¹⁸F] fluoride solution is 0.25% or less.

20. The method of claim 15, wherein the organic reactive precursor is selected from the group consisting of sugars, amino acids, proteins, nucleosides, nucleotides, small molecule pharmaceuticals, and derivatives thereof.

21. The method of claim 15, wherein the organic reactive precursor is an organic molecule having the structure X—R, wherein R is selected from the group consisting of alkyl, substituted alkyl, heterocycle, substituted heterocycle, aryl, substituted aryl, heteroaryl, and substituted heteroaryl, and X is a nucleophilic leaving group.

22. The method of claim 21, wherein X is a halogen or a pseudohalogen.

23. The method of claim 15, wherein the organic reactive precursor and the fluorine-18 fluoride solution are moved through the micro reactor using at least one pump.

24. The method of claim 15, wherein the micro reactor comprises a first microchannel segment in fluid communication with the first inlet of the micro reactor, a second microchannel segment in fluid communication with the second inlet of the micro reactor, and a third microchannel segment in fluid communication with the outlet of the micro reactor, wherein the first, second and third microchannel segments intersect.

25. The method of claim 15, wherein the fluorine-18 fluoride labeled radiochemical collected from the micro reactor is selected from the group consisting of 2-deoxy-2-[¹⁸F]fluoro-D-glucose([¹⁸F]FDG), 6-[¹⁸F]fluoro-L-3,4-dihydroxyphenylalanine ([¹⁸F]FDOPA), 6-[¹⁸F]fluoro-L-meta-tyrosine([¹⁸F]FMT), 9-[4-[¹⁸F]fluoro-3-(hydroxymethyl)butyl]guanine([¹⁸F]FHBG), 9-[(3-[¹⁸F]fluoro-1-hydroxy-2-propoxy)methyl]guanine([¹⁸F]FHPG), 3-(2′-[¹⁸F]fluoroethyl)spiperone([¹⁸F]FESP), 3′-deoxy-3′-[¹⁸F]fluorothymidine([¹⁸F]FLT), 4-[¹⁸F]fluoro-N-[2-[1-(2-methoxyphenyl)-1-piperazinyl]ethyl]-N-2-pyridinyl-benzamide([¹⁸F]p-MPPF), 2-(1-{6-[(2-[¹⁸F]fluoroethyl)(methyl)amino]-2-naphthyl}ethylidine)malononitrile([¹⁸F]FDDNP), 2-[¹⁸F]fluoro-α-methyltyrosine, [¹⁸F]fluoromisonidazole([¹⁸F]FMISO), 5-[¹⁸F]fluoro-2′-deoxyuridine([¹⁸F]FdUrd), and protected forms thereof.

26. The method of claim 15, wherein the fluorine-18 fluoride labeled radiochemical collected from the micro reactor is 2-deoxy-2-[¹⁸F]fluoro-D-glucose ([¹⁸F]FDG),6-[¹⁸F]fluoro-L-3,4-dihydroxyphenylalanine([¹⁸F]FDOPA), or a protected form thereof.

27. The method of claim 15, further comprising performing at least one additional method step in a microfluidic environment, the at least one additional method step being selected from the group consisting of deprotecting the fluorine-18 fluoride labeled radiochemical, purifying the fluorine-18 fluoride labeled radiochemical, and assaying radioactivity of the fluorine-18 fluoride labeled radiochemical.

28. A system for synthesizing a radiochemical in a microfluidic environment, the system comprising:
a micro reactor comprising a first inlet port, a second inlet port, an outlet port, and at least one microchannel in fluid communication with the first and second inlet ports and the outlet port;
a supply of a reactive precursor in fluid communication with the first inlet port of the micro reactor, the reactive precursor adapted for reaction with a radioactive isotope to form a radiochemical; and
a supply of a solution comprising a radioactive isotope in fluid communication with the second inlet port of the micro reactor.

29. The system of claim 28, wherein the supply of isotope-containing solution comprises a solution of the radioactive isotope dissolved in a polar aprotic solvent.

30. The system of claim 29, wherein the polar aprotic solvent is selected from the group consisting of acetonitrile, acetone, N,N-dimethylformamide (DMF), dimethylsulfoxide (DMSO), and hexamethylphosphoramide (HMPA).

31. The method of claim 28, wherein the supply of isotope-containing solution is a solution of a radioactive isotope selected from the group consisting of fluorine-18 fluoride, carbon-11, nitrogen-13, and oxygen-15.

32. The system of claim 28, wherein the supply of isotope is fluorine-18 fluoride in the form of a coordination compound consisting of a phase transfer catalyst and salt complex.

33. The system of claim 28, wherein the supply of reactive precursor is a supply of an organic molecule selected from the group consisting of sugars, amino acids, proteins, nucleosides, nucleotides, small molecule drugs, and derivatives thereof.

34. The system of claim 33, wherein the reactive precursor is an organic molecule having the structure X—R, wherein R is selected from the group consisting of alkyl, substituted alkyl, heterocycle, substituted heterocycle, aryl, substituted aryl, heteroaryl, and substituted heteroaryl, and X is a nucleophilic leaving group.

35. The system of claim 34, wherein X is a halogen or a pseudohalogen.

36. The system of claim 28, wherein the supply of reactive precursor is a supply of reactive precursor dissolved in a polar aprotic solvent.

37. The system of claim 28, further comprising at least one pump operatively positioned to propel the reactive precursor and the isotope-containing solution through the micro reactor.

38. The system of claim 37, comprising a first pump in fluid communication with said supply of reactive precursor and said first inlet of said micro reactor and a second pump in fluid communication with said supply of isotope-containing solution and said second inlet of said micro reactor.

39. The system of claim 28, further comprising a heat source operatively positioned to heat at least a portion of the micro reactor.

40. The system of claim 28, wherein said micro reactor is a microchip comprising a substrate having said at least one microchannel formed therein.

41. The system of claim 28, wherein said micro reactor comprises a length of capillary tubing defining said at least one microchannel.

42. The system of claim 28, wherein said micro reactor comprises a first microchannel segment in fluid communication with said first inlet of said micro reactor, a second microchannel segment in fluid communication with said second inlet of said micro reactor, and a third microchannel segment in fluid communication with said outlet of said micro reactor, wherein the first, second and third microchannel segments intersect.
